(19) United States
(12) Patent Application Publication
(10) Pub. No.: US 2014/0221871 A1
(43) Pub. Date: Aug. 7, 2014

(54) CERVICO-VAGINAL SAMPLE COLLECTION KIT

(71) Applicant: Jerome Belinson, University Hts., OH (US)

(72) Inventor: Jerome Belinson, University Hts., OH (US)

(21) Appl. No.: 13/576,029

(22) PCT Filed: Aug. 31, 2011
(86) PCT No.: PCT/US11/49922

§ 371 Date: Jul. 30, 2012

(51) Int. Cl. A61B 10/00; A61B 10/02

(52) U.S. Cl. CPC A61B 10/0096; A61B 10/02; A61B 2010/0216
USPC 600/569; 229/68.1; 435/287.1

(57) ABSTRACT

A sample collection kit (10) for detecting the presence of the human papillomavirus in samples containing cervico-vaginal cells. The sample collection kit includes a pre-addressed envelope (12) containing a sample collection device (18) and a specimen processing card (14) having two testing surfaces (40a,40b), one of which is accessible on either side of the specimen processing card. In use, the sample collection device is inserted into the subject's vagina to collect a sample of cervico-vaginal cells and then the sample collection device is wiped against one of two testing surfaces on the specimen processing card to transfer the sample of endocervical cells to the processing card. Next, the subject disposes the specimen processing card in the pre-addressed envelope and forwards the pre-addressed envelope with the specimen processing card to a medical testing facility.

### TECHNICAL FIELD OF THE INVENTION

The present invention relates to a home cervico-vaginal sample collection kit. More specifically, the present invention relates to a cervico-vaginal sample collection kit with the required components that allows a woman to conduct a cervical cancer test in the privacy of her home, and then submit the test to a laboratory for the results.

### BACKGROUND OF THE INVENTION

Statistics indicate that a large number of women die each year as a result of cervical cancer. Health professionals agree that a great proportion of these fatalities could have been avoided if the pre-cancers (cervical intraepithelial lesions) were detected or the cervical cancers had been diagnosed earlier. In an ideal situation, women would receive a medical check at short intervals so that the existence of cervical and vaginal pre-cancers could be readily diagnosed and treated. All too often the existence of abnormal cells are detected at a stage where the only action is hysterectomy or more radical cancer treatment when available. Even with the provision of proper therapy physicians have often found that because of the delay in detecting the infection and consequently the evolution of a true cancer, the available treatment is unsuccessful in halting the progress of this dreaded disease.

Unfortunately, many women are deterred from regular testing at a medical facility due to unavailability of such facility, financial burdens, time constraints, cultural issues, or personal modesty. As a result, many develop fatal cervical cancer that could otherwise be avoided with proper testing.

The critical background to this invention, known to the inventors since they have done the research and published their science (see references), is that the key element for highly effective screening actually rests with the laboratory testing procedure used when combined with self-sampling. Therefore, there is a need for a cervico-vaginal sample collection kit that is safe, simple, and as inexpensive as possible.

### SUMMARY OF THE INVENTION

A preferred embodiment discloses a cervico-vaginal sample collection kit for the detection of the presence of the human papillomavirus, the necessary cause of cervical cancer. This test kit comprises a pre-addressed envelope containing instructions, a simple sample collection device and a specimen processing card (that serves as a dry transport media) having two testing surfaces, one of which is accessible on either side of the specimen processing card.

A further embodiment discloses a method for self collecting a sample of cervico-vaginal cells from the vagina for detecting the presence of the human papillomavirus (HPV). This method includes first providing the subject with a pre-addressed envelope containing a sample collection device and a specimen processing card. Next, the subject positions the sample collection device into the vagina to collect a sample of cells and wipes the sample collection device against one of two testing surfaces on the specimen processing card to transfer the sample of endocervical cells to the processing card. Fourth, the subject disposes the specimen processing card in the pre-addressed envelope. Further, the subject forwards the pre-addressed envelope with the specimen processing card to a medical testing facility retaining the ID number that is printed on a “tear-off” from the envelope.

### BRIEF DESCRIPTION OF THE DRAWINGS

The structure, operation, and advantages of the present invention will become further apparent upon consideration of the following description taken in conjunction with the accompanying figures (FIGs.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a front view a mailing envelope incorporating the cervico-vaginal sample collection kit, in accordance with the present invention.

FIG. 2 is a rear view a mailing envelope incorporating the cervico-vaginal sample collection kit, in accordance with the present invention.

FIG. 3 is a three-dimensional front view of the testing swab, in accordance with the present invention.

FIG. 4 is a three-dimensional front view of the testing swab in a sterilized wrapping, in accordance with the present invention.

FIG. 5 is an exploded view of the specimen collecting board, in accordance with the present invention.

FIG. 6A is a top view of the specimen processing card, in accordance with the present invention.

FIG. 6B is a bottom view of the specimen processing card, in accordance with the present invention.

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

The present invention is directed to a cervico-vaginal sample collection kit and a method that enables a woman to conduct a cervical cancer-screening test that is both sensitive and specific for cervical intraepithelial neoplasia (cervical pre-cancer). The screening test is inexpensive and easy to perform because it does not require a time consuming and relatively expensive pelvic examination which requires the use of speculums and extensive clinical infrastructure. In the preferred embodiment, a home cervico-vaginal sample collection kit 10 is provided which allows a woman to test for this disease in the privacy of her home, and then simply send the results in a non-liquid, non-infectious, stable form to a laboratory for processing. All of the components required for the testing are included in the sample collection testing kit. While the cervico-vaginal sample collection kit is described as being used in a home environment, it is also within the terms of the invention to use it in clinics or any desired environment.

The disclosed embodiment is particularly advantageous because: (a) the described self-collection method is easy to manage; (b) the time and expense involved in obtaining a sample are reduced; (c) the woman can collect her own cervical/vaginal specimen in a private location which tends to increase patient compliance; (d) the described cervico-vaginal sample collection kit and method of use is affordable and accessible to those who cannot afford or are unable to obtain physician help; and (e) the specimen collected by the method and the collection kit is adaptable for screening for cervical intraepithelial neoplasia and cervical cancer. The present embodiment further provides a sample collection kit and method for detecting the presence of HPV in samples containing substantially fewer of the infected cells. As noted earlier this is documented in the sited/published work of the inventor.

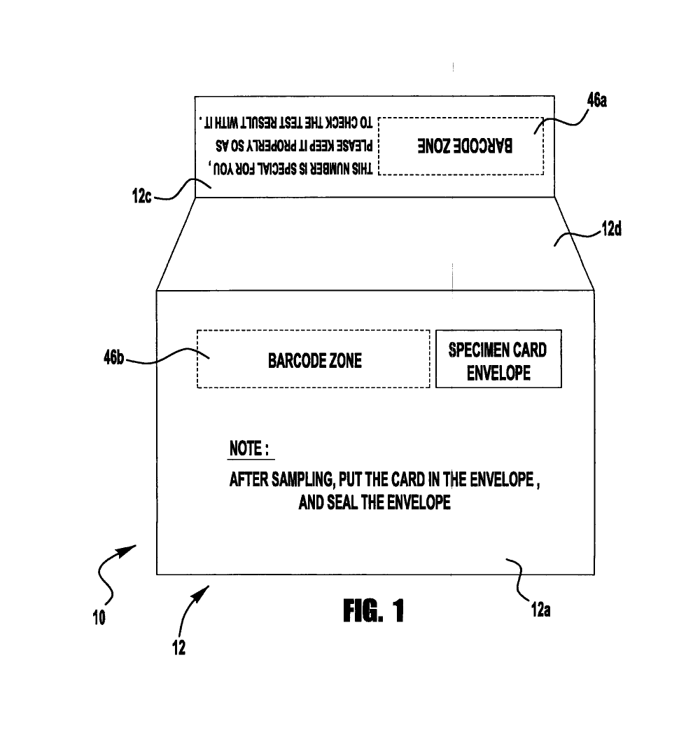

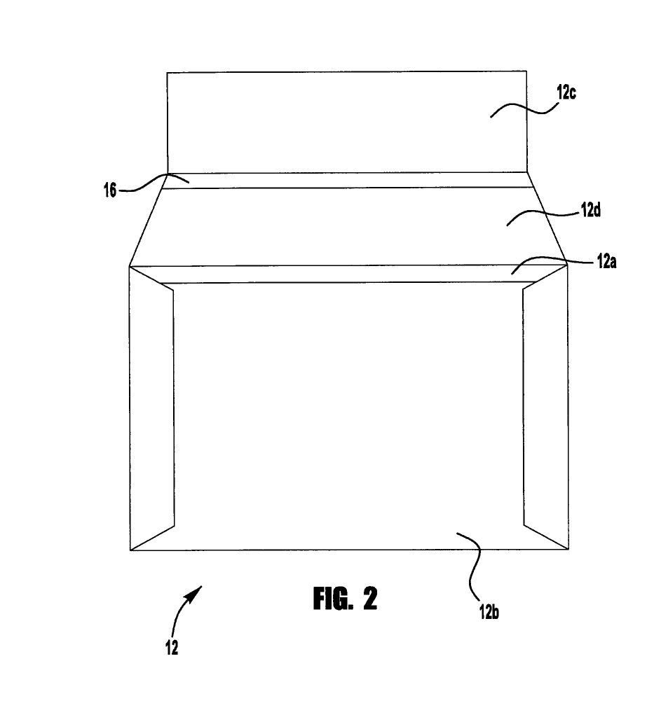

Referring to FIG. 1, the cervico-vaginal sample collection kit 10 includes an envelope 12. The front side 12a of the envelope 12 can include instructions to insert a sample collection media 14 onto which a sample has been wiped and then seal the envelope, such as with a plastic strip 16 on flap 12d, shown on the back side 12b of the envelope 12, as shown in FIG. 2. The envelope 12 includes a flap 12c with a unique barcode to be removed from the envelope and kept by the user to check the test results. Referring to FIG. 2, the sample collection media 14 onto which a sample has been wiped is inserted into the pre-addressed envelope between the front side 12a and the rear side 12b. Then the plastic strip 16 is removed and the flap 12d can be secured to the rear side 12b.

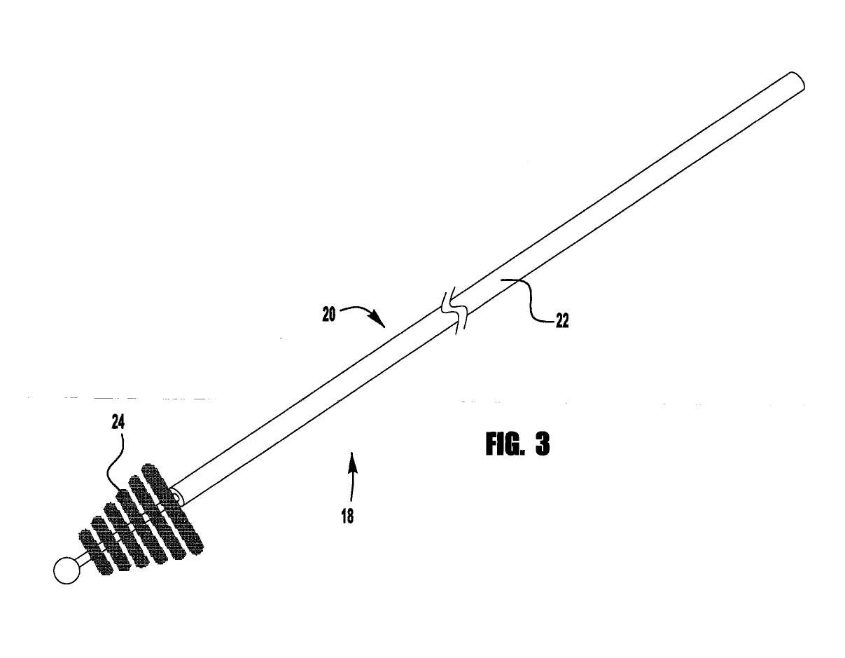

As shown in FIG. 3, a sample collection device 18 comprises a cervical sampling brush 20 that includes a longitudinal axis that runs through a tube 22 having bristles 24 at one end that extend laterally outward from the longitudinal axis. The cervical sampling brush 20 including bristles 24 and the tube 22 as a whole are called the collection device 18. The tube 22 is preferably cylindrical in shape and forms the handle portion.

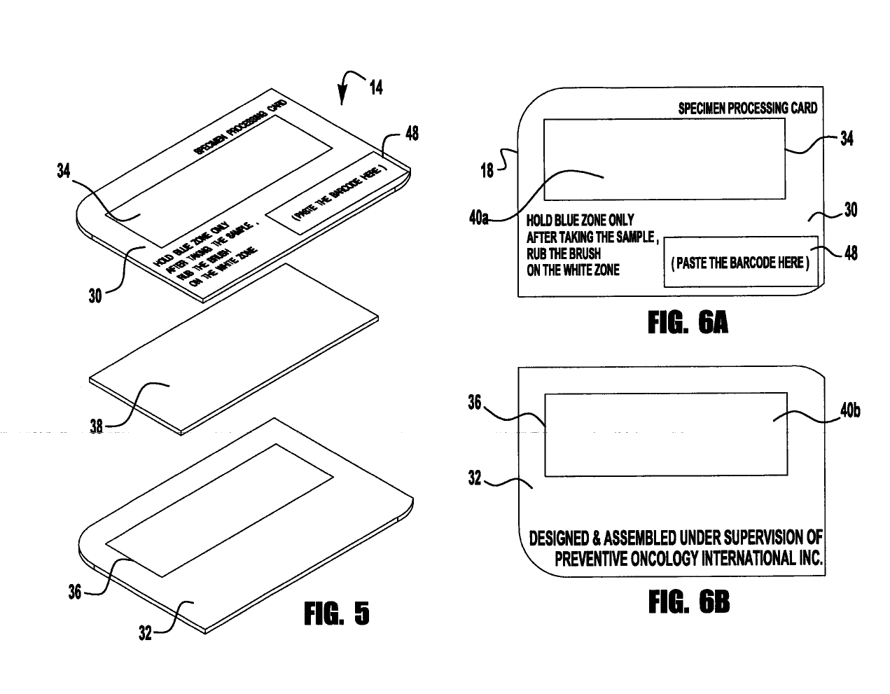

The cervical sampling brush 20 will likely be constructed of lightweight material and produced inexpensively. The woman will typically hold the brush 20 by the handle portion 22 when taking the specimen sample. After taking the test, the bristles 24 are wiped upon the transport media 14 (see FIG. 6). As part of sample collection kit 10, brush 20 will be sterilized and provided in a sterile package 26. After use, the user may simply dispose of the brush 20.

The total length of the brush 20 is approximately between 19.4 cm to 20 cm.

The diameter of the tube 22 is approximately between 0.28 cm to 0.32 cm. The bristles of brush 20 are approximately between 1.8 to 2.0 cm in length, and the diameter of the bristles is approximately 1.0-1.5 cm with smaller diameters at the tip of the bristles. The bristles can be made of any appropriate material known to one skilled in the art. The bristles are preferably made of a flexible plastic material such as polyethylene, polyurethane, polyvinyl chloride, polysiloxanes or nylon, etc. The preferred embodiment is nylon and has fiber thickness of 0.09 mm.

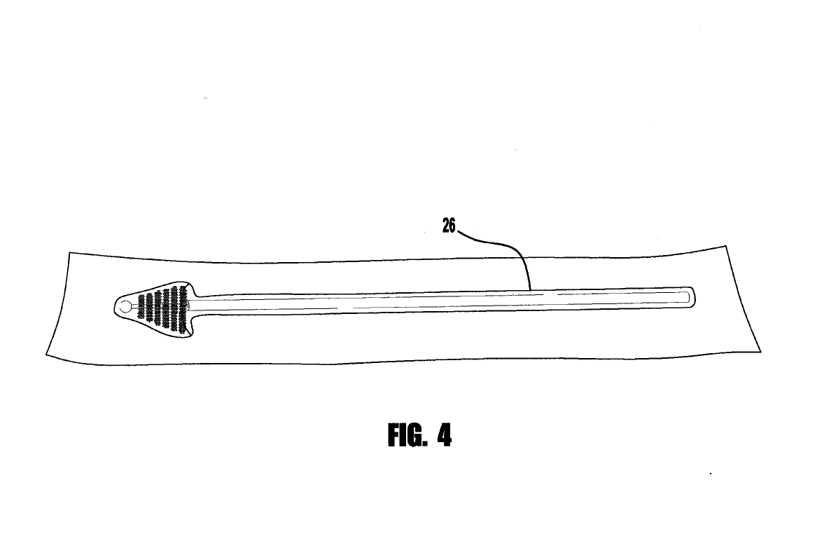

Cervico-vaginal sample collection kit 10 will include a sample collection device 18 for obtaining a self-collected cervico-vaginal sample (as seen in FIG. 3), printed instructions, a solid transport media 14 (as seen in FIGS. 5, 6A and 6B), and a pre-addressed envelope 12. This home sample collection kit 10 preempts the need for a woman to visit a medical facility to properly be tested for cervical cancer. Sample collection kit 10 has applications throughout the world. However, it would be most useful in developing countries, in which 85% of the global cases of cervical cancer reside. Using sample collection kit 10 allows for similar sensitivity and only slightly lower specificity for cervical intraepithelial neoplasia as can be accomplished by a direct endocervical specimen obtained by a physician. The implications for massive country-wide screening “events” in low and middle resource countries are profound. These findings open the possibility to screen thousands of women each day at a fraction of the cost and a fraction of the time of current paradigms.

As seen in FIGS. 5, 6A and 6b, there is a sample collection media 14 also called a specimen processing card herein. Specimen processing card 14 will be constructed of upper and lower pieces 30 and 32 of light weight cardboard stock, each having a rectangular cutout opening 34 and 36, respectively. Between the upper and lower pieces 30 and 32 of cardboard stock will be disposed a filter paper 38. The upper and lower pieces 30 and 32 of cardboard stock with the filter paper 38 therebetween can be joined together to form the specimen processing card 14 by any conventional means such as gluing the inner facing sides 30a and 32a with the filter paper therebetween. It is also within the terms of the present invention to hinge the upper and lower pieces 30 and 32 of cardboard stock together on one side. In that case, the upper and lower pieces are folded together during manufacture with the filter paper in between and slightly larger than the cutout openings 34 and 36. In either case the filter paper 38 will be exposed in the rectangular cutout openings 34 and 36. The filter paper 38 within the rectangular cutout opening 34 and 36 forms upper and lower testing surfaces 40a and 40b, along which the user swipes bristles 24 of brush 20. Having the testing surfaces on opposite sides of the specimen processing card 14 makes it easier for the user to swipe the testing surface irrespective of which side of the card is most convenient for the user. One example of a filter paper 20 is FTA elute paper made by GE Healthcare.

In use, the woman receives a pre-addressed envelope 12 containing the sterilized brush 20 in a sterile package 26, specimen processing card 14, and written instructions. The written instructions will explain how to properly utilize sample collection kit 10. The woman takes a vaginal specimen utilizing the bristles 24 of brush 20. The woman then wipes the bristles 24 along the testing surface 40a or 40b of the specimen processing card 14. The brush 20 may then be disposed of, and the removable strip with the barcode information from the barcode zone 46b disposed on the front 12a of the envelope 12 can be attached to the barcode zone 48 on the side 30 of the specimen processing card 14. Note that the barcode information can be provided on removable strips, one located in the barcode zone 46a on the flap 12c and another removable strip with barcode information can be located in the barcode zone 46b on the front side 12a of the envelope 12.

Next the specimen processing card 14 is inserted into the pre-addressed envelope 12 through an opening between the front side 12a and the rear side 12b of the envelope 12. The envelope 12 can be sealed with the adhesive under the plastic strip 16. Then the flap 12c with the barcode is separated from the envelope 12 and kept by the woman so that she can find out the results of the test. Once the results of the test are procured, the woman is notified about further medical attention that may be required. It should be noted that although testing kit 10 is exemplified for use in for cervical cancer, it has applications in other genetic, or DNA testing.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. A cervico-vaginal sample collection kit for the detection of the presence of the human papillomavirus (HPV) in samples containing cervico-vaginal cells, comprising:
a pre-addressed envelope containing components of the self-collection test kit, the components including:
a sample collection device;
a specimen processing card having two testing surfaces, one of which is accessible on either side of the specimen processing card.

2. The sample collection kit of claim 1 wherein the two testing surfaces are formed from a filter paper.

3. The sample collection kit of claim 2 wherein specimen processing card will be constructed of upper and lower pieces of paper stock, each having a cutout opening.

4. The sample collection kit of claim 3 wherein the cutout openings of the specimen processing card each have a rectangular shape.

5. The sample collection kit of claim 3 wherein the upper and lower pieces of specimen processing card and the filter paper are joined together to form a single structure.

6. The sample collection kit of claim 1 wherein the sample collection device is a sterilized cervical sampling brush.

7. The sample collection kit of claim 6 wherein the cervical sampling brush includes a handle having a longitudinal axis that runs there through and bristles at one end that extend laterally outward from the longitudinal axis.

8. The sample collection kit of claim 6 wherein the sterilized cervical sampling brush is contained within a sterile package.

9. The sample collection kit of claim 1 further including written instructions to explain how to properly utilize the test kit.

10. The sample collection kit of claim 1 wherein the pre-addressed envelope includes a flap with a unique barcode to be removed from the envelope and kept by the user to check the test results.

11. (canceled)

12. (canceled)

13. (canceled)

14. (canceled)

15. (canceled)

16. (canceled)

17. (canceled)

18. (canceled)

19. (canceled)

20. (canceled)
